(19) United States
(12) Patent Application Publication
(10) Pub. No.: US 2010/0104517 A1
(43) Pub. Date: Apr. 29, 2010

(54) Anhydrous Antiperspirant Compositions

(76) Inventor: Yan-fei Shen, Canton, MA (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/603,605

(22) Filed: Oct. 22, 2009

Related U.S. Application Data
(60) Provisional application No. 61/107,893, filed on Oct. 23, 2008.

(51) Int. Cl. A61K 9/12

(52) U.S. Cl. 424/47

(57) ABSTRACT

An anhydrous aerosol antiperspirant composition comprising a concentrate comprising at least about 40%, by weight of the concentrate, of an alcohol; an antiperspirant active dissolved in the alcohol; and at least one volatile silicone oil that is miscible in the alcohol and antiperspirant active solution and that has a viscosity of less than 10 centistokes at 25° C.

### CROSS REFERENCE TO RELATED APPLICATION

This application claims the benefit of U.S. Provisional Application No. 61/107,893, filed Oct. 23, 2008.

### FIELD OF THE INVENTION

The present invention is directed to anhydrous antiperspirant compositions.

### BACKGROUND OF THE INVENTION

Liquid antiperspirant compositions have existed for several years. Liquid compositions can be applied through use of porous applicators (e.g., roll-on products), can be sprayed with pumping technologies or can be sprayed with the use of a propellant in an aerosol system. Aerosol technology has gained favor for being both effective and relatively inexpensive. When developing aerosolized antiperspirant products, the skilled artisan faces a number of different design challenges, including, for example, clinical efficacy, skin feel, and formulation aspects that work well with standard actuators and nozzles to deliver acceptable spray rates and patterns and that do not clog the actuators of nozzles. Meeting some design challenges may result in failures for others; thus, there is room for improvement in the art.

### DETAILED DESCRIPTION OF THE INVENTION

The present invention may be understood more readily by reference to the following detailed description of illustrative and preferred embodiments. It is to be understood that the scope of the claims is not limited to the specific components, methods, conditions, devices, or parameters described herein, and that the terminology used herein is not intended to be limiting of the claimed invention. Also, as used in the specification, including the appended claims, the singular forms “a,” “an,” and “the” include the plural, and reference to a particular numerical value includes at least that particular value, unless the context clearly dictates otherwise. When a range of values is expressed, another embodiment includes from the one particular value and/or to the other particular value. Similarly, when values are expressed as approximations, by use of the antecedent basis “about,” it will be understood that the particular values form another embodiment. All ranges are inclusive and combinable.

The antiperspirant compositions of the present invention can comprise, consist essentially of, or consist of, the described components as well as optional ingredients described herein. As used herein, “consisting essentially of” means that the composition or component may include additional ingredients, but only if the additional ingredients do not materially alter the basic and novel characteristics of the claimed compositions or methods.

All percentages, parts and ratios are based upon the total weight of the topical compositions of the present invention and all measurements made are at 25° C., unless otherwise specified. All such weights as they pertain to listed ingredients are based on the active level and, therefore, do not include carriers or by-products that may be included in commercially available materials, unless otherwise specified.

The present invention is directed to anhydrous antiperspirant compositions. The term “anhydrous” as used herein, unless otherwise specified, refers to those materials or compositions that are substantially free of added water. As it pertains to the compositions of the present invention, this means that the compositions are essentially free of added water. The term “anhydrous”, however, as used herein can also mean that the composition contains water but that the water is isolated. The term “anhydrous” as used herein generally means that the material or composition preferably contains less than about 1%, less than about 0.5%, or zero percent, by weight of free or added water.

The antiperspirant compositions generally include a concentrate and a propellant to define a final composition. Alcohol is a predominant component of the concentrates provided herein. Useful alcohols include C₁-C₃ alcohols, with the preferred alcohol being ethanol. In exemplary embodiments, the alcohol is employed at a concentration level of from at least about 40%, 50% or 55% to about 80%, by weight of the concentrate.

An antiperspirant active is dissolved in the alcohol, at a level of from about 1% to about 15%, by weight of the concentrate. Various antiperspirant actives can be employed, including, for example, aluminum chloride, aluminum chlorohydrate, aluminum chlorohydrex, aluminum chlorohydrex PG, aluminum chlorohydrex PEG, aluminum dichlorohydrate, aluminum dichlorohydrex PG, aluminum dichlorohydrex PEG, aluminum sesquichlorohydrate, aluminum sesquichlorohydrex PG, aluminum sesquichlorohydrex PEG, aluminum sulfate, aluminum zirconium octachlorohydrate, aluminum zirconium octachlorohydrex GLY, aluminum zirconium pentachlorohydrate, aluminum zirconium pentachlorohydrex GLY, aluminum zirconium tetrachlorohydrate, aluminum zirconium trichlorohydrate, aluminum zirconium tetrachlorohydrate GLY, and aluminum zirconium trichlorohydrate GLY. In one preferred embodiment, aluminum chlorohydrex PG is the chosen antiperspirant active.

The antiperspirant concentrates also include an oil or a mixture of two or more oils. Useful oils include, for example, volatile silicone oils and non-volatile organic oils. “Volatile silicone”, as used herein, refers to those silicone materials that have measurable vapor pressure under ambient conditions. Non-limiting examples of suitable volatile silicones are described in Todd et al., “Volatile Silicone Fluids for Cosmetics”, Cosmetics and Toiletries, 91:27-32 (1976). The volatile silicone may be a cyclic silicone having from at least about 3 silicone atoms or from at least about 5 silicone atoms but no more than about 7 silicone atoms or no more than about 6 silicone atoms. For example, volatile silicones may be used which conform to the formula:

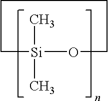

wherein n is from about 3 or from about 5 but no more than about 7 or no more than about 6. These volatile cyclic silicones generally have a viscosity of less than about 10 centistokes at 25° C. Suitable volatile silicones for use herein include, but are not limited to, Cyclomethicone D5 (commercially available from G. E. Silicones); Dow Corning 344, and Dow Corning 345 (commercially available from Dow Corning Corp.); and GE 7207, GE 7158 and Silicone Fluids SF-1202 and SF-1173 (available from General Electric Co.). SWS-03314, SWS-03400, F-222, F-223, F-250, F-251 (available from SWS Silicones Corp.); Volatile Silicones 7158, 7207, 7349 (available from Union Carbide); MASIL SF-V (available from Mazer) and combinations thereof. Suitable volatile silicone oils also include linear silicone oils such as, for example, DC200 (1 cSt), DC200 (0.65 cSt), and DC2-1184, all of which are available from Dow Corning Corp. In exemplary embodiments, the volatile silicone oil has a viscosity of less than 10 centistokes at 25° C.

Non-volatile organic, emollient oils may also be employed. A representative, non-limiting list of emollient oils includes CETIOL CC (dicaprylyl carbonate), CETIOL OE (dicaprylyl ether), CETIOL S (diethylhexylcyclohexane), and CETIOL B (dibutyl adipate), all of which are available from Cognis, and LEXFEEL 7 (neopentyl glycol diheptanoate) from Inolex. In exemplary embodiments, the organic emollient oils have a viscosity of less than 50 centistokes at 25° C. The term “organic emollient oil” as used herein means silicon-free emollient oils that are liquid at 25° C., and that are safe and light to skin and can be miscible with volatile silicone oils (as described above) and the antiperspirant active-alcohol solution in the concentration ranges described below.

The oil or mixture of oils is generally included in the concentrate formulas at a level of from about 5% to about 45%, by weight of the concentrate. This viscosity ranges noted above in connection with the different classes of oil can facilitate desired spray rates and patterns, and can help minimize nozzle clogging. To provide desired skin feel, minimal nozzle clogging, and good concentrate stability, the ratio of alcohol to volatile silicone oil is preferably greater than 1.0, 1.35, or 1.5. And in embodiments having both a volatile silicone oil and an organic emollient oil, the ratio of alcohol to total oil is preferably greater than or equal to about 0.90. The oils in exemplary embodiments are miscible with the alcohol and antiperspirant active solution. Although various levels of miscibility are acceptable, the oils are preferably miscible enough with the alcohol and antiperspirant active solution to yield a concentrate having a clear appearance.

Antiperspirant compositions of the present invention may include one or more fragrance/perfume materials. In one preferred embodiment, the composition includes a fragrance material comprising a plurality of different perfume raw materials. Typical perfume levels in the present invention are 0.25 to 5%. Nonlimiting examples of fragrance materials include any known fragrances in the art or any otherwise effective fragrance materials. Typical fragrances are described in Arctander, “Perfume and Flavour Chemicals (Aroma Chemicals)”, Vol. I and II (1969) and Arctander, “Perfume and Flavour Materials of Natural Origin” (1960). U.S. Pat. No. 4,322,308, issued to Hooper et al., Mar. 30, 1982 and U.S. Pat. No. 4,304,679, issued to Hooper et al., Dec. 8, 1981 disclose suitable fragrance materials including, but not limited to, volatile phenolic substances (such as iso-amyl salicylate, benzyl salicylate, and thyme oil red), essence oils (such as geranium oil, patchouli oil, and petitgrain oil), citrus oils, extracts and resins (such as benzoin siam resinoid and opoponax resinoid), “synthetic” oils (such as Bergamot™ 37 and Bergamot™ 430, Geranium™ 76 and Pomeransol™ 314), aldehydes and ketones (such as B-methyl naphthyl ketone, p-t-butyl-A-methyl hydrocinnamic aldehyde and p-t-amyl cyclohexanone), polycyclic compounds (such as coumarin and beta-naphthyl methyl ether), esters (such as diethyl phthalate, phenylethyl phenylacetate, non-anolide 1:4).

Suitable fragrance materials may also include esters and essential oils derived from floral materials and fruits, citrus oils, absolutes, aldehydes, resinoides, musk and other animal notes (e.g., natural isolates of civet, castoreum and musk), balsamic, and alcohols (such as dimyrcetol, phenylethyl alcohol and tetrahydromuguol). For example, the antiperspirant compositions may comprise fragrances selected from the group consisting of decyl aldehyde, undecyl aldehyde, undecylenic aldehyde, lauric aldehyde, amyl cinnamic aldehyde, ethyl methyl phenyl glycidate, methyl nonyl acetaldehyde, myristic aldehyde, nonalactone, nonyl aldehyde, octyl aldehyde, undecalactone, hexyl cinnamic aldehyde, benzaldehyde, vanillin, heliotropine, camphor, para-hydroxy phenolbutanone, 6-acetyl 1,1,3,4,4,6 hexamethyl tetrahydronaphthalene, alpha-methyl ionone, gamma-methyl ionone, amyl-cyclohexanone, and mixtures thereof. Fragrance materials other than those listed above may also be employed.

The antiperspirant compositions can also include residue-masking agents to reduce the appearance of white residue arising from the antiperspirant active and structurant (if present) employed in the product. Exemplary residue-masking agents include isostearyl isostearate, glycereth-7-benzoate, C12-C15 alkyl benzoate, octyldodecyl benzoate, isostearyl lactate, isostearyl palmitate, benzyl laurate, laureth 4, laureth 7, oleth 2, PEG 4, PEG 12, isopropyl myristate isopropyl palmate, butyl stearate, polyethylene glycol methyl ethers, PPG 2 ceteareth 9, PPG 2 isodeceth 12, PPG 5 butyl ether, PPG 14 butyl ether, PPG 15 butyl ether, PPG 53 butyl ether, octyldodecanol, polydecene, mineral oil, petrolatum, phenyltrimethicone, dimethicone copolyol, and mixtures thereof. One preferred concentration level of the optional residue-masking agent is from about 3% to about 10%, by weight of the concentrate. But other levels may also be used.

The antiperspirant concentrate is combined with a propellant before or after filling into a suitable aerosol container. The final antiperspirant composition generally includes less than 10%, 5%, or 1%, by weight of the composition, of a propellant. Suitable propellants of the present invention include, but are not limited to, butane, isobutane, propane, dimethyl ether, 1, 1 difluoroethane, nitrogen (N₂), carbon dioxide (CO₂), and mixtures thereof. In one preferred embodiment, the propellant is nitrogen.

The following examples further describe and demonstrate embodiments within the scope of the present invention. The examples are given solely for the purpose of illustration and are not to be construed as limitations of the present invention as many variations thereof are possible without departing from the spirit and scope of the invention.

TABLE 1
Antiperspirant Concentrates (to be combined with a propellant
to form an aerosol antiperspirant composition).

| Ingredient | A | B | C | D | E | F |
|---|---|---|---|---|---|---|
| Aluminum Chlorohydrex-PG | 10.0 | 10.0 | 10.0 | 10.0 | 10.0 | 10.0 |
| Ethanol | 45.0 | 55.0 | 55.0 | 55.0 | 55.0 | 55.0 |
| Trisiloxane (viscosity = 1 cSt) | 45.0 | 35.0 | | | | |
| Dimethicone (viscosity = 10 cSt) | | | 35.0 | | | |
| Cyclopentasiloxane (viscosity = 4 | | | | 35.0 | | |
| cSt) | | | | | | |
| Cyclohexasiloxane (viscosity = 6.8 | | | | | 35.0 | |
| cSt) | | | | | | |
| Dicaprylyl Carbonate (viscosity = | | | | | | 35.0 |
| 2.5-6.5 cSt) | | | | | | |
| Ethanol:Silicone Oil Ratio | 1.0 | 1.57 | 1.57 | 1.57 | 1.57 | |
| Initial Clarity | Slightly | Clear | Slightly | Clear | Clear | Slightly |
| | cloudy | | cloudy | | | cloudy |
| Stable | No | Yes | No | Yes | Yes | No |

The values are shown on a by weight of the concentrate basis.

TABLE 2
Antiperspirant Concentrates (to be combined with a propellant
to form an aerosol antiperspirant composition).

| Ingredient | G | H | I | J | K |
|---|---|---|---|---|---|
| Aluminum Chlorohydrex-PG | 10.6 | 12.05 | 12.03 | 10.85 | 9.49 |
| Ethanol | 44.34 | 38.85 | 41.76 | 43.62 | 56.39 |
| Trisiloxane (viscosity = 1 cSt) | | | | | 15.04 |
| Dimethicone (viscosity = 10 cSt) | | | | | |
| Cyclopentasiloxane (viscosity = 4 | 16.96 | 18.0 | 18.22 | 16.22 | 9.59 |
| cSt) | | | | | |
| Cyclohexasiloxane (viscosity = 6.8 | 14.67 | 15.0 | 11.95 | 13.10 | 35.0 |
| cSt) | | | | | |
| Organic Emollient Oil (2- | 13.43 | 16.1 | 16.05 | 16.22 | |
| octyldodecanol) | | | | | |
| Organic Emollient Oil (Dicaprylyl | | | | | 9.49 |
| either) | | | | | |
| Ethanol:Silicone Oil Ratio | 1.4 | 1.18 | 1.38 | 1.49 | 2.29 |
| Ethanol:Total Oil (Silicone Oil + | 0.98 | 0.79 | 0.90 | 0.96 | 1.65 |
| Organic Emollient Oil) Ratio | | | | | |
| Initial Clarity | Clear | Clear | Clear | Clear | Clear |
| Stable | Yes | No | Yes | Yes | |

The values are shown on a by weight of the concentrate basis.

The above examples can be made as follows: add aluminum antiperspirant active to ethanol, and mix and heat at 70° C. until a clear solution is obtained; cool the solution to room temperature; and then add the remaining ingredients.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1) An anhydrous aerosol antiperspirant composition, comprising:
(a) a concentrate comprising:
i) at least about 40%, by weight of the concentrate, of an alcohol;
ii) an antiperspirant active dissolved in the alcohol; and
iii) at least one volatile silicone oil that is miscible in the alcohol and antiperspirant active solution and that has a viscosity of less than 10 centistokes at 25° C.,
wherein the ratio of alcohol to the at least one volatile silicone oil is greater than 1.0; and
(b) less than 10% of a propellant mixed with the concentrate to form the anhydrous aerosol antiperspirant composition.

2) The composition of claim 1, wherein the alcohol is ethanol.

3) The composition of claim 1, wherein the concentration comprises at least about 50%, by weight, of an alcohol.

4) The composition of claim 1, wherein the antiperspirant active comprises an aluminum chlorohydrate material.

5) The composition of claim 1, wherein the concentrate includes from about 1% to about 15%, by weight, of the antiperspirant active.

6) The composition of claim 1, wherein the ratio of alcohol to volatile silicone oil is greater than or equal to 1.5.

7) The composition of claim 1, wherein the propellant comprises nitrogen.

8) An anhydrous aerosol antiperspirant composition, comprising:
(a) a concentrate comprising:
i) at least about 40%, by weight of the concentrate, of an alcohol;
ii) an antiperspirant active dissolved in the alcohol;
iii) a volatile silicone oil that is miscible in the alcohol and antiperspirant active solution and that has a viscosity of less than 10 centistokes at 25° C.; and
iv) an organic emollient oil,
wherein the ratio of alcohol to volatile silicone oil is greater than 1.0, and wherein the ratio of alcohol to organic emollient oil is greater than or equal to about 0.90; and
(b) less than 10% of a propellant mixed with the concentrate to form the anhydrous aerosol antiperspirant composition.

9) The composition of claim 8, wherein the alcohol is ethanol.

10) The composition of claim 8, wherein the antiperspirant active comprises an aluminum chlorohydrate material.

11) The composition of claim 8, wherein the concentrate includes from about 1% to about 15%, by weight, of the antiperspirant active.

12) The composition of claim 8, wherein the propellant comprises nitrogen.
